(19) United States
(12) Patent Application Publication
(10) Pub. No.: US 2006/0177520 A1
(43) Pub. Date: Aug. 10, 2006

(54) Compositions and methods for reducing cytotoxicity and inhibiting angiogenesis

(75) Inventors: Yuanjin Tao, Fremont, CA (US); Lily C. Yang, Los Altos, CA (US)

Correspondence Address: MORRISON & FOERSTER LLP, 755 PAGE MILL RD, PALO ALTO, CA, 94304-1018, US

(73) Assignee: TheraLife, Los Altos, CA (US)

(21) Appl. No.: 11/202,954

(22) Filed: Aug. 11, 2005

Related U.S. Application Data
(63) Continuation of application No. 10/140,747, filed on May 6, 2002, now abandoned.
(60) Provisional application No. 60/288,722, filed on May 4, 2001.

(51) Int. Cl. A61K 36/65; A61K 31/355; A61K 31/34; A61K 31/205; A61K 33/04; A61K 31/375; A61K 36/254; A61K 33/32; A61K 36/48

(52) U.S. Cl. 424/641; 424/725; 424/702; 424/757; 424/771; 424/728; 424/773; 514/554; 424/764; 514/458; 514/474

(57) ABSTRACT

Compositions and methods for reducing cytotoxicity are provided. Compositions and methods for inhibiting angiogenesis are provided. The compositions comprise herbals, natural nutritional supplements, minerals and/or vitamins.

### CROSS-REFERENCE TO RELATED APPLICATIONS

This application is a continuation application of U.S. Ser. No. 10/140,747, filed May 6, 2002 which claims the priority benefit of U.S. Provisional application 60/288,722, filed May 4, 2001, the disclosures of which are incorporated herein by reference in their entirety.

### TECHNICAL FIELD

This invention relates to compositions and methods for reducing cytotoxicity. More specifically, it relates to nutraceutical compositions and methods for reducing cytotoxicity, preferably cytotoxicity of kidney and liver cells.

### BACKGROUND ART

Drug toxicity is a major problem in drug development efforts, and is a major cause of failure of otherwise promising drug leads. Because therapeutic substances, such as pharmaceutical drugs, are usually foreign to the body, or are provided at levels not commonly found in the body or a particular site in the body, damage to cells exposed to these substances is a serious problem in drug development and/or usage. Cytotoxicity of cells of the liver and kidney is particularly prevalent, most likely because of the primary function of these organs in metabolism and waste removal, respectively.

Various herbal substances have been studied for their potential in protecting against cellular damage. Jiang & Qian, Chung Kuo Yao Li Hsueh Pao (1995), 16(5):399-402; Leung et al., Neurochem. Res. (1991), 16(6):687-92; Yoshikawa et al., Chem. Pharm. Bull (Tokyo) (1997), 45(6):1039-45; Liu et al., J. Ethnopharmacol. (1994), 42(3):183-91; Qi, Chung Hsi I Chieh Ho Tsa Chih (1991), 11(2):102-4, 69; Fu, Chung Kuo Chung Hsi I Chieh Ho Tsa Chih (1992), 12(4):228-9, 198; Zee-Cheng, Methods Find. Exp. Clin. Pharmacol. (1992), 14(9):725-36; Zee-Cheng, Methods Find. Exp. Clinic. Pharmacol. (1992), 14(9):725-36; Wang & Han, Yao Hsueh Hsueh Pao (1993), 28(8):572-6; Haraguchi et al., Bioorg. Med. Chem. (1998), 6(3):339-47; Haraguchi et al., J. Pharm. Pharmacol. (2000), 52(2):219-23; Ju et al., Yao Hsueh Hsueh Pao (1989), 24(11):807-12; Hu et al., Biomed. Environ. Sci. (1999), 12(1):10-4; Luper, Altern. Med. Rev. (1999), 4(3):178-88; Shim et al., Planta Med. (2000), 66(1):40-3; Acharya et al., Indian J. Med. Res. (1993), 98(-HD-):69-74; Yokozawa et al., Phytomedicine (2000), 6(6):439-45; Mokhova et al., FEBS Lett. (1991), 289(2):187-9; Visentin et al., J. Pharmacol. Exp. Ther. (1995), 275(2):1069-75. The substances studied were found to provide varying degrees of cell protective functions.

In view of the seriousness and prevalence of cellular toxicity associated with exposure to otherwise therapeutic, and thus beneficial, compositions, it would be advantageous to have compositions and methods that are capable of reducing cytotoxicity when administered in conjunction with said otherwise beneficial compositions. The invention described and claimed in this specification presents such compositions and methods.

All references cited herein, including patent applications and publications, are incorporated by reference in their entirety.

### DISCLOSURE OF THE INVENTION

Compositions and methods for reducing cytotoxicity are provided. The compositions comprise substances in quantities that are effective for reducing cytotoxicity. In a preferred embodiment, the compositions of the invention reduce cytotoxicity associated with cellular exposure to a second composition. Preferably, a second composition is a therapeutic composition. Also provided are methods of reducing cytotoxicity comprising administration of a composition of the invention to an individual. In some embodiments, said cytotoxicity is associated with exposure to a second composition (which is preferably a therapeutic composition).

In yet another aspect, compositions and methods for inhibiting angiogenesis and/or angiogenic diseases are also provided. Methods for inhibiting angiogenesis comprising administration of the claimed compositions to an individual in need thereof are provided.

Accordingly, in one aspect, the invention provides a composition for reducing cytotoxicity, said composition comprising two substances selected from the group consisting of a member of the Glycyrrhiza genus (such as gancao), a member of the paeonia genus (such as baishaoyao), a member of the panax (such as shanqi) or eleutherococus genus, and L-carnitine. In preferred embodiments, said cytotoxicity is associated with exposure to a second composition (which is preferably a therapeutic composition).

In another aspect, the invention provides a composition for reducing cytotoxicity, said composition comprising three substances selected from the group consisting of a member of the Glycyrrhiza genus (such as gancao), a member of the paeonia genus (such as baishaoyao), a member of the panax (such as shanqi) or eleutherococus genus, and L-carnitine. In preferred embodiments, said cytotoxicity is associated with exposure to a second composition (which is preferably a therapeutic composition).

In another aspect, the invention provides a composition for reducing cytotoxicity, said composition comprising a member of the Glycyrrhiza genus (such as gancao), a member of the paeonia genus (such as baishaoyao), a member of the panax (such as shanqi) or eleutherococus genus, and L-carnitine. In preferred embodiments, said cytotoxicity is associated with exposure to a second composition (which is preferably a therapeutic composition).

In still another aspect, the invention provides a composition selected from the group consisting of the compositions of the aspects of the invention described in the preceding paragraphs, further comprising at least one (preferably 1, 2, 3, 4, 5, 6, 7 or 8) substances selected from the group consisting of a member of the Daemonorops genus (such as xuejie), a member of the corydalis genus (such as yanhusuo), white willow bark, black cohosh root, vitamin E, vitamin C and a mineral (such as zinc and selenium).

In yet another aspect, the invention provides a method of making a composition of the invention, said method comprising combining two or more (for example, 2, 3 or 4) substances (preferably in an effective amount) selected from the group consisting of a member of the Glycyrrhiza genus (such as gancao), a member of the paeonia genus (such as baishaoyao), a member of the panax (such as shanqi) or eleutherococus genus, and L-carnitine. In some embodiments, the method further comprises combining said two or more substances with at least one (for example, 1, 2, 3, 4, 5, 6, 7 or 8) substance (preferably in an effective amount) selected from the group consisting of a member of the Daemonorops genus (such as xuejie), a member of the corydalis genus (such as yanhusuo), white willow bark, black cohosh root, vitamin E, vitamin C and a mineral (such as zinc and selenium). In some embodiments, said combining is by mixing (such as by stirring, agitation or vibration). In some embodiments, the substances are packaged in the form of capsules, preferably in size “0”, “00”, “000”, “1”, “2”, “3” or “4.” In yet other embodiments, the substances are combined in powder form, preferably to at least 30%, 60%, or 90% mixture consistency, or to homogeneity.

In another aspect, the invention provides a method for reducing cytotoxicity, said method comprising administering a first composition selected from the aspects and embodiments of the invention described in the preceding paragraphs to said individual, whereby said cytotoxicity is reduced. In some embodiments, said cytotoxicity is associated with exposure to a second composition. In some embodiments of this aspect of the invention, the first composition further comprises at least one (for example, 1, 2, 3, 4, 5, 6, 7 or 8) substance selected from the group consisting of a member of the Daemonorops genus (such as xuejie), a member of the corydalis genus (such as yanhusuo), white willow bark, black cohosh root, vitamin E, vitamin C and a mineral (such as zinc and selenium). In one embodiment, the cytotoxicity associated with exposure to a second composition is of liver cells. In another embodiment, the cytotoxicity associated with exposure to a second composition is of kidney cells. In some embodiments, the second composition is a therapeutic composition.

### MODES FOR CARRYING OUT THE INVENTION

The present invention discloses compositions comprising two or more substances that in combination are effective for reducing cytotoxicity, preferably cytotoxicity associated with cellular exposure to another composition. The invention further provides methods for reducing cytotoxicity comprising administering the compositions of this invention to an individual.

The invention also provides methods for inhibiting angiogenesis and methods for treating diseases associated with unwanted and/or uncontrolled angiogenesis. These methods comprise administering an effective amount of a claimed composition to an individual, i.e., an amount sufficient to inhibit angiogenesis.

Definitions

The term “cytotoxicity”, and variations thereof, as used in this specification, refers to an unintended or undesirable alteration in the normal state of a cell. The normal state of a cell is generally that which is manifested or exists prior to the cell's exposure to a cytotoxic composition, agent and/or condition. Generally but not necessarily, a cell that is in a normal state is one that is in homeostasis. An unintended or undesirable alteration in the normal state of a cell can be manifested in the form of, for example, cell death under conditions wherein a cell is normally viable (for example, non-programmed cell death), a decrease in proliferative capability, a decrease in cellular integrity such as membrane integrity, a decrease in metabolic activity, and/or a decrease in developmental capability.

The phrase “reducing cytotoxicity”, and variations thereof, refers to reduction in degree or frequency of unintended or undesirable alterations in the normal state of a cell upon exposure to a cytotoxic composition, agent and/or condition. The phrase can refer to reducing the degree of cytotoxicity in an individual cell that is exposed to a cytotoxic composition, agent and/or condition, or to reducing the number of cells of a population that exhibit cytotoxicity when the population of cells is exposed to a cytotoxic composition, agent and/or condition.

A “second composition,” as used herein, generally refers to a composition that, when administered to an individual, is known or suspected to be associated with cytotoxicity. Cytotoxicity can be caused directly or indirectly by the second composition. A “second composition”, as used herein, refers to a single compound or substance, or a mixture of two or more compounds or substances. Preferably, a second composition is a therapeutic composition. A “therapeutic composition” is a composition administered to achieve a treatment or therapeutic effect. A therapeutic composition can be, for example, a composition comprising a pharmaceutical compound such as a drug.

“Angiogenesis,” as used herein, refers to the generation or growth of new blood vessels to cells or into tissue organs or tumors, as is understood in the art. “Anti-angiogenesis” or “anti-angiogenic activity” refers to depression, suppression and/or inhibition of angiogenesis. With regard to anti-angiogenesis, an “effective amount,” “therapeutically effective amount” or “anti-angiogenic” amount refer to an amount of a composition effective to depress, suppress or inhibit angiogenesis or result in amelioration of symptoms associated with an angiogenic disease. The desired result can be either a subjective relief of a symptom(s) or an objectively identifiable improvement in the recipient of the composition, a decrease in the vascularization of endothelial cells or a decrease in the rate of angiogenesis as noted by a clinician or other qualified observer.

The term “treating,” “treatment,” and variations thereof, as used in this specification, refers to an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a therapeutic composition.

A “treatment effect” or “therapeutic effect” is manifested if there is a change in the condition being treated, as measured by the criteria constituting the definition of the terms “treating” and “treatment.” There is a “change” in the condition being treated if there is at least 10% improvement, preferably at least 25%, more preferably at least 50%, even more preferably at least 75%, and most preferably at least 100%. The change can be based on improvements in the severity of the treated condition in an individual, or on a difference in the frequency of improved conditions in populations of individuals with and without treatment with the therapeutic compositions with which the compositions of the present invention are administered in combination.

An “effective amount” is an amount of a composition or substance(s) sufficient to reduce cytotoxicity after one or more administrations of that amount. An effective amount can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

“Co-administering” or “co-administration” of compositions, and “administered in conjunction with,” as used herein, refers to the administration of a composition of the present invention and a second composition within a certain time period. The time period is preferably 12 hours, more preferably 6 hours, still more preferably 3 hours. These terms most preferably mean the compositions are administered together.

“Individual,” as used herein, refers to a vertebrate, preferably a mammal, more preferably a human.

A “genus,” as used herein, refers to the botanical classification term as understood in the art. As used herein, it can also include botanical entities that are closely related to a genus (but are not officially classified under said genus) with respect to their relevant function(s) in the compositions of the invention.

“Xuejie,” as used herein, refers to extracts of xuejie. It is also known as sanguis draconis and Daemonorops droco B1. A member of the genus Daemonorops is preferably selected from the group consisting of Daemonorops draco B1 (xuejie), Dracaena cambodiana Pierre, Dracaena cinnabari Balf, Dracaena draco Linn., Dracaena schizantha Baker, Dracaena dihynophyllus, Dracaena micracanthus Becc., Dracaena propinqus Becc., Dracaena draconcellus Becc., Dracaena motleyi Becc., Dracaena sparsiflorus Becc., Pterocarpus draco Linn., Croton draco Schlecht, Croton hibiscifolius kunth, Croton sanguifluus H. B. et K Nov. and Croton gossypiifolius Vahl.

“Yanhusuo,” as used herein, refers to extracts of yanhusuo. It is also known as Rhizoma corydalis yanhusuo and Corydalis yanhusuo W. T. Wang. A member of the genus Corydalis is preferably selected from the group consisting of Corydalis yanhusuo W. T. Wang (yanhusuo), Corydalis amabilis Migo, Corydalis amabigua Cham. et Schlecht. Var amurensts Maxim, Corydalis turtschaninovii Bess., Corydalis schangini (Pall.) B., Corydalis glaucescens Rgl. and Corydalis ledebouriana Kar. Et Kir.

“Baishaoyao,” as used herein, refers to extracts of baishaoyao. It is also known as Radix paeoniae lactiflorae and paeonia lactiflora Pall. A member of the genus Paeonia is preferably selected from the group consisting of Paeonia lactiflora Pall. (Bai shao) and paeonia veitchii Lynch.

“Shanqi,” as used herein, refers to extracts of shanqi. It is also known as Radix Notoginsheng and Panax notoginsheng (Burk.) F. H. Chen. A member of the genus Panax is preferably selected from the group consisting of Panax notoginseng (Burk.) F. H. Chen (Shanqi), Panax pseudo-ginseng Wall.var.notoginseng (Burk) Hoo et Tseng, Panax ginseng C. A. Mey (P.schinseng Nees) and Panax guinquefolium L. A member of the genus eleutherococus is preferably Acanthopanax sentocosus (Rupr. et Maxim.) or Eleutherococus senticosus (Rupr. et Maxim.).

“Gancao,” as used herein, refers to extracts of gancao. It is also known as Radix glycyrrhizae uralensis, Glycyrrhiza uralensis Fischer or licorice root. A member of the glycyrrhiza genus is preferably selected from the group consisting of Glycyrrhiza uralensis Fisch (Gan cao), Glycyrrhiza glabra L., Glycyrrhizainflata Batal., Glycyrrihiza korshiskyi G. Hrig., Glycyrrhiza aspera Pall and Glycyrrhiza yunnanensis Cheng f. et L. K. Tai.

“White willow bark,” as used herein, refers to extracts of white willow bark. It is also known as Saliz alba caerulea.

“Black cohosh root,” as used herein, refers to extracts of black cohosh root. It is also known as rhizome of Cimicifuga racemosa or Cimicifuga racemosa.

“L-carnitine,” as used herein, refers to the biochemical molecule known as such by persons of skill in the art. It is generally known as a zwitterionic compound formed from lysine. It also refers to related molecules such as L-acetyl-carnitine.

“Vitamin C,” as used herein, refers to ascorbic acid and salts thereof.

“Vitamin E,” as used herein, refers to D alpha-tocopherol, preferably in succinate form.

“Zinc,” as used herein, refers to the form(s) of the mineral known to persons of skill in the art to be therapeutically effective in the body of the individual. It is preferably provided as zinc gluconate.

“Selenium,” as used herein, refers to the form(s) of the mineral known to persons of skill in the art to be therapeutically effective in the body of the individual. It is preferably provided as selenium aminoate.

“Extract,” as used herein, refers to the substances obtained from the specified source plant, or parts thereof (for e.g., root, bark, leaves). Any method of extraction that yields extracts that retain the biological activity of the substances contained in the extract source can be used to produce extracts used in this invention. Preferably, the ingredients of the compositions of the present invention are extracted as an aqueous solution. The extraction is preferably performed under conditions of high pressure, preferably from 0.5 to 12 bar, more preferably 1 to 10 bar, most preferably 3 to 7 bar, and preferably at elevated temperatures (preferably within a range of 15° C. to 120° C., more preferably 30C. to 100° C., most preferably 45° C. to 75° C.). The extract is preferably treated to yield a form suitable for mixing of two or more substances. The form is preferably a dried powder. The powder form is yielded from preferably at least about a 1:10, more preferably at least about a 1: 8, most preferably at least about a 1:5 concentrate of the starting solution. Concentration to powder form is preferably achieved by evaporation to yield a dried powder form. The extracts used in this invention can also be obtained from commercial sources such as Sun Ten Laboratories (Irvine, Calif.), Qualiherb (Cerritos, Calif.), Mayway (Oakland, Calif.), Ming Tong Herb (Oakland, Calif.) and Acta (Sunnyvale, Calif.). It is understood that any method or conditions known in the art to yield extracts comparable in effectiveness in reducing cytotoxicity to those produced by the preceding preferred extraction method can be used for the purposes of this invention.

Formulation of the Composition

Each substance contained in the compositions provided by this invention is provided in an amount that lies within specific quantitative ranges herein disclosed to be effective for reducing cytotoxicity.

According to the present invention, an effective amount of a composition comprises preferably 50 mg to 1000 mg, more preferably 200 mg to 800 mg, most preferably 400 mg to 600 mg of a member of the Glycyrrhiza genus (such as gancao); preferably from 50 mg to 1000 mg, more preferably 200 mg to 800 mg, most preferably 400 mg to 600 mg of a member of the Paeonia genus (such as baishaoyao); preferably 200 mg to 3000 mg, more preferably 500 mg to 2000 mg, most preferably 800 mg to 1500 mg of a member-of the Panax (such as shanqi) or Eleutherococus genus (such as Acanthopanax senticosus or Eleutherococus senticosus); and/or preferably 30 mg to 600 mg, more preferably 120 mg to 500 mg, most preferably 250 mg to 400 mg L-carnitine.

In the various embodiments of the invention, the compositions further comprise preferably from 50 to 1500 mg, more preferably 300 mg to 1200 mg, most preferably 600 mg to 900 mg of a member of the Daemonorops genus (such as xuejie); and/or preferably 50 mg to 1000 mg, more preferably 200 mg to 800 mg, most preferably 400 mg to 600 mg of a member of the Corydalis genus (such as yanhusuo); preferably 5 mg to 150 mg, more preferably 10 mg to 120 mg, most preferably 75 mg to 100 mg white willow bark; preferably 5 mg to 150 mg, more preferably 40 mg to 120 mg, most preferably 75 mg to 100 mg black cohosh root; preferably 10 mg to 250 mg, more preferably 80 mg to 300 mg, most preferably 120 mg to 200 mg vitamin E; preferably 20 mg to 400 mg, more preferably 80 mg to 300 mg, most preferably 120 mg to 200 mg vitamin C; preferably 10 mg to 80 mg, more preferably 20 mg to 60 mg, most preferably 30 mg to 50 mg zinc; and preferably 5 mg to 60 mg, more preferably 10 mg to 50 mg, most preferably 20 mg to 40 mg selenium.

According to this invention, the compositions can be formulated in whatever form that retains the efficacy of the compositions for reducing cytotoxicity. Preferably, the compositions are packaged in the form of capsules. The capsules are preferably of size “0”, “00”, “000”, “1”, “2”, “3” or “4.” A preferred method for packaging into capsules involves mixing substances (extracts, vitamin and minerals) that are preferably in powder form. The substances are preferably mixed to at least 30%, more preferably to at least 60%, even more preferably to at least 90% mixture consistency, and most preferably to homogeneity. The substances in powder form are provided in the initial mixture at ratios according to the effective quantities disclosed above. Methods for mixing the substances are known in the art, including, but not limited to, stirring, agitation or vibration achieved manually or through the aid of a machine. A preferred mixing machine is a V-mixer, preferably of 100 to 1400-liter size, more preferably of 150 to 1300-liter size, and most preferably of 200 to 1200-liter size. Preferably, the resulting powder mixture is filtered to screen out particulates (i.e., anything that a person of skill in the art would recognize to be larger than powder size). A preferred filter is a 1/20-inch particle size filter. Preferably, the filtered mixture is packaged into capsules according to the weight desired for each capsule. Preferably, the capsule is of size “00”. The weight of mixture per capsule is preferably from 5 mg to 1000 mg, more preferably 100 mg to 800 mg, even more preferably 400 mg to 700 mg. It is understood that other physical forms of the compositions of this invention suitable for administration to an individual can also be used, including, for example, tablets, salves or liquids, as long as the compositions can be delivered to the target tissues in the body where the compositions in the preferred form described above exert their effects.

The ingredients of the compositions can be mixed with pharmaceutically acceptable solvents, excipients and/or filler substances. These materials are known in the art, and are described in sources such as Remington's Pharmaceutical Sciences, 18th Edition, Mack Publishing (1990).

Administration of Compositions of the Present Invention

Compositions in any of the forms described above can be administered by any method known to one of skill in the art, but oral administration is preferred. The compositions are preferably administered in capsule form.

An effective amount of a composition is provided preferably in from 1 to 8 administrations, more preferably in from 2 to 6 administrations, and most preferably in from 3 to 5 administrations. Administration of an effective amount is preferably completed within 24 hours. A composition can be ingested alone, or with any other substance, such as a liquid, that aids ingestion of the compositions. Ingestion of the compositions can be before or after food consumption.

A composition of the invention can be administered in conjunction with a second composition, which is generally a composition that is known or suspected to be associated with cytotoxicity of cells exposed to it. Preferably, the second composition is a therapeutic composition. For example, the present compositions can be administered in conjunction with compositions that are effective in treating eye discomfort syndromes, pains and/or discomfort in joints such as the wrist, pains and/or discomfort in the neck and/or shoulder, or pains and/or discomfort in the back and leg.

Other medical conditions for which the compositions of the present invention are particularly useful include post-trauma muscle spasms, bleeding (internal and external), sciatic pain, swelling and bleeding due to injury and bruising, arthritis, conditions associated with chemotherapy, and microbial (such as viral and bacterial) diseases. The present compositions can reduce any cytotoxicity associated with compositions used to treat these conditions.

A second composition may or may not be provided in the same form as the compositions of the invention. Thus, for example, the composition of the invention may be provided in capsule form, and the second composition is provided in aerosol form. A composition of the invention and a second composition may be administered by the same route or different routes of administration. Thus, for example, one may be administered orally while another is administered by injection, or one is administered by spraying onto a mucosal surface while another is introduced orally.

The timing of administration of the composition of the invention and a second composition can be varied according to the needs of the individual, or in accordance with the empirical determination or experience of the health care professional or individual. In some embodiments, the composition of the invention is administered prior to administration of the second composition. In these embodiments, the composition of the invention is administered preferably at least 7 days, more preferably at least 5 days, even more preferably at least 3 days, and most preferably at least 0.5 day prior to administration of the second composition. In some embodiments, the composition of the invention is administered preferably at least 12 hours, more preferably at least 9 hours, even more preferably at least 6 hours, and most preferably at least 3 hours prior to administration of the second composition. In other embodiments, the composition of the invention is administered after the administration of the second composition. In these embodiments, the composition of the invention is administered preferably at least 7 days, more preferably at least 5 days, even more preferably at least 3 days, and most preferably at least 0.5 day after administration of the second composition. In some of these embodiments, the composition of the invention is administered preferably at least 12 hours, more preferably at least 9 hours, even more preferably at least 6 hours, and most preferably at least 3 hours after administration of the second composition. In some embodiments, the composition of the invention and the second composition are administered at the same time. In still other embodiments, the composition of the invention is administered before, with and after the administration of the second composition.

Reduction in cytotoxicity can be measured in any of a number of ways known in the art. In one embodiment, reduction in cytotoxicity can be assessed based on degree and/or frequency of occurrence of cytotoxicity-associated side effects. In another embodiment, reduction in cytotoxicity can be indicated by an increase in efficacy of a therapeutic composition administered in conjunction with a composition of the invention compared to the efficacy of the therapeutic composition when not administered in conjunction with a composition of the invention. In another embodiment, reduction in cytotoxicity is indicated when a reduced amount of a therapeutic composition is required to achieve a particular degree of therapeutic effect when it is administered in conjunction with a composition of the invention than when it is not. In yet another embodiment, reduction in cytotoxicity is assessed by measuring amount of cellular toxicity using assays known in the art, which include standard laboratory techniques such as dye exclusion, detection of morphologic characteristics associated with cell viability, injury and/or death, and measurement of enzyme and/or metabolic activities associated with the cell type of interest.

The present invention also relates to the discovery that the compositions of the invention are useful for inhibiting angiogenesis and, in turn, for treating diseases associated with unwanted and/or uncontrolled angiogenesis. The particular dosage of the compositions of the invention to inhibit angiogenesis and/or angiogenic diseases will depend upon the severity of the condition, the route of administration and related factors that will be decided by an attending physician. As anti-angiogenic compositions, the compositions of the invention are useful in the treatment of a variety of diseases associated with angiogenesis. For example, the anti-angiogenic compositions are useful in the treatment of both primary and metastatic solid tumors and may also be useful in treating solid tumors that arise from hematopoietic malignancies such as leukemias as well as in the treatment of lymphomas. In addition, these compositions may be useful in the prevention of metastasis from these tumors either when used alone or in combination with radiotherapy and/or other chemotherapeutic agents. Further uses include the treatment and prophylaxis of autoimmune diseases including, but not limited to, rheumatoid, immune and degenerative arthritis; various ocular diseases including, but not limited to, diabetic retinopathy and retinal neovascularization due to macular degeneration; and other diseases characterized by excessive or abnormal stimulation of endothelial cells including, but not limited to, intestinal adhesions and Crohn's disease.

Inhibition of angiogenesis can be assessed by any of a variety of methods known in the art. For example, reduction in tumor size, tumor growth, tumor burden and/or reduced rate of tumor metastasis as based on physical exam, laboratory parameters, tumor markers, radiographic findings or findings based on other imaging systems. Compositions suitable for use in the anti-angiogenesis methods of the invention can readily be identified using in vitro and in vivo screening assays known in the art. See, for example, U.S. Pat. Nos. 5,801,146 6,150,407. Such assays may screen for the ability of a particular composition to inhibit angiogenesis or the vascularization of endothelial cells in vitro and in vivo. For instance, the chick embryo chorioallantoic membrane (CAM) assay, in which the test composition is implanted on the chorioallantoic membrane of chick embryos, can be used to screen a given composition for its ability to inhibit vascularization. A clear avascular zone around the implanted source of the composition is an indication of anti-angiogenic activity. In another example, a human microvascular endothelial cell assay (HMVEC) or a human umbilical vein microvascular endothelial cells (HUMVEC) proliferation assays can be used to assess the anti-angiogenesis properties and/or efficacy of the compositions.

The following Example is provided to illustrate, but not limit, the invention.

### EXAMPLES

### Example 1

An Illustrative Example of the Formulation of A Single “00” Capsule, and the Production Thereof

A composition capable of reducing cytotoxicity contains substances in the indicated quantities as listed in Table 1.

TABLE 1

| SUBSTANCE [Commercial Source] | AMOUNT (mg) |
|---|---|
| Xuejie [Min Tong Herb, Oakland, CA] | 133 |
| Yanhusuo [Mayway, Oakland, CA] | 83 |
| Baishaoyao [Qualiherb, Cerritos, CA] | 83 |
| Shanqi [Sun Ten, Irvine, CA] | 250 |
| Gancao [Qualiherb, Cerritos, CA] | 83 |
| White willow bark [Acta, Sunnyvale, | 10 |
| CA] | |
| Black cohosh root [Acta, Sunnyvale, | 10 |
| CA] | |
| L-carnitine [Acta, Sunnyvale, CA] | 50 |
| Vitamin E (D Alpha-Tocopherol | 21.19 |
| succinate) [Acta, Sunnyvale, CA] | |
| Vitamin C [Acta, Sunnyvale, CA] | 30 |

Capsules containing the composition above are manufactured according to the method used by a commercial manufacturer, such as Acta (Sunnyvale, Calif.). Briefly, the substances listed above, in powder form and obtained from the commercial sources such as those indicated in Table 1, are mixed in input amounts in accordance to the ratio of the substances in the composition as a whole. Mixing is accomplished with a V-mixer, grinding for 15 to 30 minutes, at a speed of 15 to 30 rpm (rounds per minute), to produce a homogenous mixture of the input substances. Particulates (non-powder forms) are then filtered out with a 1/20-inch particle size filter that separates particulates from the powder. 671 mg of the filtered mixture is then packaged into each size “00” capsule.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

What is claimed is:

1. A composition for reducing cytotoxicity, said composition comprising two substances selected from the group consisting of gancao, baishaoyao, shanqi and L-carnitine.

2. A composition for reducing cytotoxicity, said composition comprising three substances selected from the group consisting of gancao, baishaoyao, shanqi and L-carnitine.

3. A composition for reducing cytotoxicity, said composition comprising gancao, baishaoyao, shanqi and L-carnitine.

4. The composition of any of claims 1-3, further comprising at least one substance selected from the group consisting of xuejie, yanhusuo, white willow bark, black cohosh root, vitamin E, vitamin C, zinc and selenium.

5. A method for reducing cytotoxicity in an individual associated with cellular exposure to an administered therapeutic composition, said method comprising administering to the individual an effective amount of a composition comprising two substances selected from the group consisting of licorice root, peonia, noto ginseng and L-carnitine, whereby said cytotoxicity is reduced.

6. A method for reducing cytotoxicity in an individual associated with cellular exposure to an administered therapeutic composition, said method comprising administering to the individual an effective amount of a composition comprising three substances selected from the group consisting of licorice root, peonia, noto ginseng and L-carnitine, whereby said cytotoxicity is reduced.

7. A method for reducing cytotoxicity in an individual associated with cellular exposure to an administered therapeutic composition, said method comprising administering to the individual an effective amount of a composition comprising licorice root, peonia, noto ginseng and L-carnitine, whereby said cytotoxicity is reduced.

8. The method of any of claims 5-7, wherein said composition further comprises at least one substance selected from the group consisting of xuejie, yanhusuo, white willow bark, black cohosh root, vitamin E, vitamin C, zinc and selenium.

9. The method of any of claims 5-7, wherein said cytotoxicity is to liver or kidney cells.

10. The method of claim 8, wherein said cytotoxicity is to liver or kidney cells.

11. A method of making a composition for reducing cytotoxicity associated with cellular exposure to an administered therapeutic composition, said method comprising combining at least two substances selected from the group consisting of gancao, baishaoyao, shanqi and L-carnitine.
